(19) United States
(12) Patent Application Publication
(10) Pub. No.: US 2008/0114058 A1
(43) Pub. Date: May 15, 2008

(54) Use Of Lignan Compounds For Treating Or Preventing Inflammatory Disease

(76) Inventors: Jae-Kwan Hwang, Gyeonggi-do (KR); Do-Un Kim, Gyeonggi-do (KR); Jae-Youn Chung, Seoul (KR); Hee-Chul Chung, Gyeonggi-do (KR); Kyu-Lee Han, Seoul (KR)

Correspondence Address: JHK LAW, P.O. BOX 1078, LA CANADA, CA, 91012-1078, US

(21) Appl. No.: 11/813,590

(22) PCT Filed: Jan. 6, 2006
(86) PCT No.: PCT/KR06/00065

§ 371 Date: Oct. 5, 2007

(30) Foreign Application Priority Data
Jan. 7, 2005 (KR) 10-2005-0001761

(51) Int. Cl. A61K 31/05; A61K 31/335

(52) U.S. Cl. 514/462; 514/733

(57) ABSTRACT

The present invention relates to the use of lignan compounds for treating or preventing an inflammatory disease. More particularly, it relates to a pharmaceutical composition for the treatment or prevention of an inflammatory disease, comprising a lignan compound represented by Formula I, as well as a treating method and the use of an inflammatory disease using the lignan compound. The lignan compound has the effect of inhibiting inflammatory reactions by inhibiting the production or expression of inflammation mediators NO, iNOS, PGE₂, COX-2 and TNF-α. Accordingly, the lignan compound or a Myristica fragrans extract will be highly useful for the treatment or prevention of an inflammatory disease.

### CROSS-REFERENCE TO RELATED APPLICATIONS

This application claims priority to Korean Patent Application No. 10-2005-0001761, filed on Jan. 7, 2005, the contents of which are hereby incorporated by reference.

### FIELD OF THE INVENTION

The present invention relates to the use of lignan compounds for treating or preventing an inflammatory disease. More particularly, it relates to a pharmaceutical composition for the treatment or prevention of an inflammatory disease, comprising a lignan compound represented by Formula I, as well as a treating method and the use of an inflammatory disease using the lignan compound.

### BACKGROUND OF THE INVENTION

Inflammatory reactions result from tissue (cell) injury or infection by foreign pathogens and show a series of complex physiological responses such as enzyme activation, inflammation mediator release, body fluid infiltration, cell movement and tissue destruction, and external symptoms such as erythema, edema, pyrexia, pain and etc., in which various inflammation-mediating factors and immune cells in local blood vessels and body fluids are involved. Also, in some cases, these inflammation reactions result in acute inflammation, granuloma, and chronic inflammations such as rheumatoid arthritis and osteoarthritis (Goodwin J. S. et al., J. Clin. Immunol., 9: 295-314, 1989).

Among enzymes having important effects on blood coagulation and inflammation, cyclooxygenase (hereinafter, referred to as ‘COX’) produces two main products, i.e., prostaglandin and thromboxane. Prostaglandin is an unsaturated fatty acid having various physiological activities and acts as local hormones or cell function regulators in the human body, such as inflammation and pain transmission, vasodilation, body temperature regulation, and gastric secretion stimulation (Marnett, L. J. et al., J. Biol. Chem., 274: 22903-22906, 1999). COX-1 plays an important role in the maintenance of cell homeostasis by maintaining normal physiological responses, such as gastrointestinal tract protection, renal blood flow regulation and platelet aggregation. Meanwhile, in a process wherein inflammation caused by external stimulus is transmitted, inducible isoenzyme COX-2 is temporarily expressed to release an excessive amount of prostaglandin at the site where inflammation occurs. Prostaglandin causes erythema, edema and pain, the main symptoms of inflammation, and has an activity of increasing the action of endogenous inflammatory mediator histamine, and the like. Thus, the inhibition of prostaglandin production at inflammatory sites can give much help in the treatment of inflammation.

Currently commercially available non-steroidal anti-inflammatory drugs (NSAIDs) aspirin, indomethacin, naproxen, ibuprofen and the like show anti-inflammatory effects by suppressing prostaglandin production through the inhibition of activity of COX-2 enzyme (Meade E. A. et al., J. Biol. Chem., 268: 6610, 1993). However, these NSAID drugs have problems in that they also inhibit COX-1 from playing an important role in maintaining the normal function of gastrointestinal tract and renal platelet, in addition to inhibiting COX-2 temporarily expressed by inflammatory stimulus, and thus cause severe side-effects, such as gastrointestinal tract bleeding and renal failure (Surh Y. J. et al., Mutation Research 480-481: 243-268, 2001). Accordingly, it is very important from an industrial point of view to find a natural substance that provides anti-inflammatory action while minimizing side effects.

Meanwhile, lignan refers to a group of natural compounds comprising n-phenyl propane bound to the i-position of the n-propyl side chain and is widely distributed in nature. There have been studies on the various physiological activities of lignan, such as blood glucose-lowering action, anticancer action, anti-asthmatic action and whitening action. For example, it was reported that lignans isolated from sesame, such as sesamin, episesamin, sesaminol, sesamolin and episesaminol, have anti-inflammatory effects (Korean Patent Laid-Open Publication No. 1997-7001043), and lignan compounds isolated from Magnoliae flos can be used as anti-asthmatic agents (Korean Patent Registration No. 0263439). Moreover, macelignan is a typical lignan compound found in Myristica fragrans (Tuchinda P. et al., Phytochemistry, 59: 169-173, 2002), and was reported to have various activities, such as the activation of caspase-3 inducing apoptosis (Park B. Y. et al., Biol. Pharm. Bull., 27(8): 1305-1307, 2004), and antioxidant action (Sadhu, S. K. et al., Chem. Pharm. Bull., 51(9): 595-598, 2003). However, there is still no report on the anti-inflammatory activity of lignan compounds, including macelignan.

### DETAILED DESCRIPTION OF THE INVENTION

Technical Problem

Accordingly, the present inventors have conducted a long-term investigation to find a naturally derived compound having anti-inflammatory activity and, as a result, found that a lignan compound isolated and purified from a Myristica fragrans extract shows excellent anti-inflammatory activity, thereby completing the present invention.

It is an object of the present invention to provide the use of lignan compounds for treating or preventing inflammatory disease.

Technical Solution

To achieve the above object, in one aspect, the present invention provides a pharmaceutical composition for the treatment or prevention of an inflammatory disease, comprising a lignan compound represented by Formula I or a pharmaceutically acceptable salt thereof as an active ingredient:

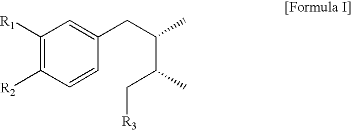

wherein R₁ and R₂ are each independently a C₁₋₅ alkoxy group or a hydroxyl group, and R₃ is

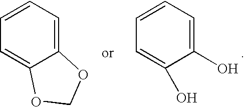

In another aspect, the present invention provides a method for preventing or treating an inflammatory disease, comprising administering to a subject in need thereof an effective amount of a lignan compound represented by Formula I or a pharmaceutically acceptable salt thereof.

In still another aspect, the present invention provides the use of a lignan compound of Formula I for preparing a pharmaceutical composition for the prevention or treatment of an inflammatory disease.

As used herein, the term “effective amount” refers to the amount of the inventive lignan compound, which can effectively treat an inflammatory disease when being administered to a subject.

Also, as used herein, the term “subject” encompasses mammals, particularly animals including human beings. The subject may be a patient in need of treatment.

Hereinafter, the present invention will be described in detail.

The present invention is characterized by providing a novel use of a lignan compound isolated and purified from a Myristica fragrans extract.

The lignan compound according to the present invention is represented by Formula I:

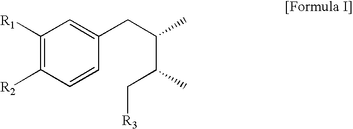

wherein R₁ and R₂ are each independently a C₁₋₅ alkoxy group or a hydroxyl group, and R3 is

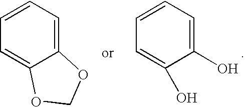

In the present invention, preferable the lignan compound may be macelignan of Chemical Formula 1, i.e., [(8R, 8′S)-7-(3,4-methylenedioxyphenyl)-7′-(4-hydroxy-3-methoxyphenyl)-8,8′-dimethylbutane)], wherein R₁ is a methoxy group, R₂ is a hydroxyl group, and R₃ is

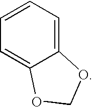

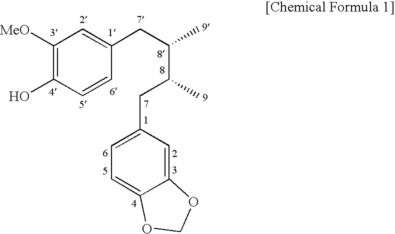

The lignan compound according to the present invention may be used in the form of a salt, and preferably a pharmaceutically acceptable salt. Preferably, the salt is the acid-addition salt formed by a pharmaceutically acceptable free acid. The free acid used in the present invention may be organic acids and inorganic acids. The organic acids include, but are not limited to, citric acid, acetic acid, lactic acid, tartar acid, maleic acid, fumaric acid, formic acid, propionic acid, oxalic acid, trifluoroacetic acid, benzoic acid, gluconic acid, methane sulfonic acid, glycolic acid, succinic acid, 4-toluene sulfonic acid, glutamic acid and aspartic acid. Also, the inorganic acids include, but are not limited to, hydrochloric acid, bromic acid, sulfuric acid and phosphoric acid.

The inventive lignan compound can be obtained from a plant or part of a plant according to any conventional method for extracting and isolating substance. Stems, roots or leaves are suitably dehydrated and macerated or only dehydrated in order to obtain the desired extract, which is then purified using any conventional purification method known to a person skilled in the art. Synthetic compounds or their derivatives corresponding to the lignan compound represented by Formula I are generally commercially available substances or they may be manufactured using any known synthetic method.

The inventive lignan compound represented by Formula I may be isolated and purified from Myristica fragnance Houtt (Jung Yun Lee et al., Kor. J. Pharmacogn. 21(4): 270-273, 1990; Masao Hattori et al., Chem. Pharm. Bull., 34(9): 3885-3893, 1986; Masao Hattori et al., Chem Pharm. Bull., 35(2): 668-674, 1987). Preferably, it may be isolated and purified from nutmeg or aril. The nutmeg refers to the ripe fruit of Myristica fragnance or a seed contained in the fruit. Moreover, the inventive lignan compound may also be isolated and purified from oil obtained by squeezing nutmeg. Also, it may be isolated and purified from Myristica argentea Warb, another member of the Myristicaceae family (Filleur, F. et al., Natural Product Letters, 16: 1-7, 2002). In addition, it may also be isolated and purified from Machilus thunbergii (Park B. Y. et al., Biol. Pharm. Bull., 27(8): 1305-1307, 2004), and Leucas aspera (Sadhu, S. K. et al., Chem. Pharm. Bull., 51(9): 595-598, 2003).

An extraction solvent for isolating the inventive lignan compound may be water or a C₁-C₆ organic solvent. Preferred examples of the extraction solvent may include purified water, methanol, ethanol, propanol, isopropanol, butanol, acetone, ether, benzene, chloroform, ethyl acetate, methylene chloride, hexane, cyclohexane, petroleum ether and the like, which can be used alone or a mixture thereof. More preferably, methanol or hexane may be used. The isolation and purification of the inventive lignan compound from an extract of Myristica fragnance may be performed by one or combination of, for example, column chromatography and high-performance liquid chromatography (HPLC), packed with various synthetic resins, such as silica gel or activated alumina. However, the method for isolating and extracting the active ingredient needs not to be limited to these chromatography techniques.

As such, the inventive lignan compound may be used in the form of an isolated and purified compound or in the form of an extract containing the compound. As described above, the inventive lignan compound may be used in the form of an extract of the seed or fruit of Myristica fragnance or an aril extract, or in the form of oil obtained by squeezing the seed of Myristica fragnance. As described above, the extract can be obtained by extracting Myristica fragnance with water or a C₁-C₆ organic solvent. Preferably, the extract may be an extract of the seed of Myristica fragnance, namely, a nutmeg extract.

The inventive lignan compound has anti-inflammatory activity by inhibiting various substances that mediate inflammatory reactions.

Nitric oxide (NO), which is a substance involved in nervous system transmission, relaxation of blood vessel, and cell-mediated immune responses, is produced from L-arginine by NOS (nitric oxide synthase) (Nathan and Xie, 1994; Alderton et al., 2001). Particularly when macrophages are stimulated by IFN-γ or LPS (lipopolysaccaride), iNOS (inducible nitric oxide synthase) will be expressed and a large amount of NO will be produced by the iNOS. It was shown that the inventive lignan compound concentration-dependently inhibited the production of NO in machrophages and the expression of iNOS involved in the production of NO (see FIGS. 8 and 9).

Also, COX-2 is a substance involved in inflammatory responses in vivo and produces inflammatory prostaglandin (PG). The expression of COX-2 is induced by endotoxin LPS secreted by bacteria, and inflammatory cytokines IL-1, TNF-α, IFN-γ and the like. The inventive lignan compound has the effects of inhibiting the expression of COX-2 and also inhibiting the production of PGE₂ (prostaglandin E2), a member of PE family, in a concentration-dependent manner (see FIGS. 10 and 11).

TNF-α (tumor necrosis factor α) is a major mediator of acute inflammatory reactions caused by gram-negative bacteria and other infectious microorganisms. Macrophages stimulated by LPS increase the synthesis of TNF-α. In biological action, TNF-α acts on leukocytes and epithelial cells at low concentrations so as to induce acute inflammation. At moderate concentrations, it mediates systemic inflammatory reactions, and at high concentrations, it causes death by pathological abnormality of septic shock. Also, TNF-α produces fever by increasing the synthesis of PG, and causes vascular plugging by inhibiting the expression of trombomodulin (Abbas and Lichtman, “Cellular and Molecular Immunology” the fifth edition. pp. 247-253, 2003). The inventive lignan compound has the effect of inhibiting the production of TNF-α in macrophages and human monocytic cells (see FIGS. 12 and 13).

The present inventors applied the inventive lignan compound locally on the ears of rats having edema induced by treatment with TPA (12-O-tetradecanoylphorbol-13-acetate). As a result, the inventive lignan compound inhibited the formation of edema in a concentration-dependent manner and showed a percent edema inhibition higher than that of currently commercially available anti-inflammatory drug indomethacin (see Table 2). Also, the present inventors prepared creams comprising the lignan compound and applied the creams locally on the ears of rats. As a result, the creams greatly inhibited the formation of edema (see Table 4).

Meanwhile, the present inventors applied Myristica fragnance extracts (methanol and hexane crude extracts) locally on the ears of rats having edema by treatment with TPA. As a result, it could be observed that the extracts inhibited the formation of edema in a concentration-dependent manner (see Table 5).

These results suggest that the inventive lignan compound shows excellent anti-inflammatory action by inhibiting not only COX-2, but also various factors that mediate inflammation reactions. Also, the results indicate that the Myristica fragnance extract can show the same anti-inflammatory effect even by itself. The anti-inflammatory activities of the inventive lignan compound represented by Formula I and of the Myristica fragnance extract were found for the first time in the present invention.

In view of the fact that currently commercially available non-steroidal anti-inflammatory drugs mostly show anti-inflammatory effects by inhibiting the activity of COX-2 enzyme, it can be seen that the inventive lignan compound can be used as an anti-inflammatory drug having a higher effect than those of the prior anti-inflammatory drugs.

Accordingly, the present invention provides a pharmaceutical composition for the treatment or prevention of an inflammatory disease, which contains the lignan compound of represented by Formula I or a pharmaceutically acceptable salt thereof as an active ingredient. Also, the present invention provides a pharmaceutical composition for the treatment or prevention of an inflammatory disease, which contains the Myristica fragnance extract as an active ingredient. The preparation of the Myristica fragnance extract is performed in the same manner as described above.

Furthermore, the present invention provides a method for preventing or treating an inflammatory disease, the method comprising administering to a subject in need thereof an effective amount of the compound of represented by Formula I or a pharmaceutically acceptable salt thereof.

In addition, the present invention provides the use of the lignan compound of represented by Formula I for preparing a pharmaceutical composition for the prevention or treating an inflammatory disease.

The inventive lignan compound or a pharmaceutically acceptable salt thereof can be administered orally or parenterally and used in form of common drug formulations. The common drug formulations may be prepared using fillers, thickeners, binders, wetting agents, disintegrants, and diluents such as surfactants, or excipients. Solid formulations for oral administration include tablets, pills, powders, granules, and capsules and are prepared by combining the lignan compound or the Myristica fragnance extract with at least one excipient, for example, starch, calcium carbonate, sucrose, lactose or gelatin. Also, except the simple excipient, lubricant such as magnesium stearate or talc may be used. Examples of liquid formulations for oral administration include suspensions, liquids, emulsions and syrups. The liquid formulations may comprise a simple diluent such as water, liquid paraffin, and various excipients, for example, humectants, sweet agents, aromatic agents and preservatives. Examples of pharmaceutical formulations for parenteral administration include sterilized aqueous solutions, non-aqueous solutions, suspensions, emulsions, freeze-dried preparations, ointments and creams. The non-aqueous solutions and suspensions may be prepared using propylene glycol, polyethylene glycol, vegetable oils such as olive oil, and injectable esters such as ethyloleate.

Also, the inventive lignan compound or a pharmaceutically acceptable salt thereof may be administered by parenteral rotes, including subcutaneous, intravenous, intramuscular or intraperitoneal injection. For parenteral administration, the lignan compound of represented by Formula I or the Myristica fragnance extract may be mixed with a stabilizer or buffer in water to prepare a solution or suspension, which may then be provided as ampules or vials each containing a unit dosage form. The dosage units can contain, for example, 1, 2, 3, or 4 times of an individual dose or ½, ⅓ or ¼ times of an individual dose. An individual dose preferably contains the amount of an effective drug which is given in one administration and which usually corresponds to a whole, a half, a third or a quarter of a daily dose.

The inventive lignan compound of represented by Formula I or the Myristica fragnance extract can be administered in an effective dosage of 0.1-50 mg/kg, and preferably 1-10 mg/kg, 1-3 times a day. The dosage of the inventive compound or extract may vary depending on, for example, the body weight, age, sex, health condition, diet, time of administration, method of administration, excretion rate and disease severity for a certain patient.

The inventive lignan compound was tested for toxicity in oral administration to rats, and as a result, it was observed that the 50% lethality (LD50) was more than 2,000 mg/kg.

Particularly, the inventive pharmaceutical composition comprising the lignan compound or the Myristica fragnance extract can be formulated in the form of drugs for skin application, i.e., ointments and creams, and it may be properly combined by the form of drugs in the range of 0.001-10.0 wt %, and preferably 0.005-5.0 wt %, based on the total weight of a formulation. If the composition is used in an amount of less than 0.005 wt %, it will provide low anti-inflammatory activity, and if it is added in an amount of more than 10 wt %, it will show no significant difference in anti-inflammatory activity only increasing an additive.

The present invention, the term “inflammatory disease” refers to a disease involving an inflammation caused by various stimulative factors, such as NO, iNOS, COX-2, PGE₂ and TNF-α, that induce a series of inflammatory reactions. Examples of the inflammatory disease include, but are not limited to, common inflammatory symptoms such as edema, inflammatory bowel disease, peritonitis, osteomyelitis, cellulitis, pancreatitis, trauma causing shock, bronchial asthma, allergic rhinitis, cystic fibrosis, acute bronchitis, chronic bronchitis, acute bronchiolitis, chronic bronchiolitis, osteoarthritis, gout, spondyloarthropathy, ankylosing spondylitis, Reiter's syndrom, psoriatic arthropathy, spondylitis associated with inflammatory bowel disease, juvenile arthropathy, juvenile ankylosing spondylitis, reactive arthropathy, infectious arthritis, post-infectious arthritis, gonococcal arthritis, tuberculous arthritis, viral arthritis, fungal arthritis, syphilitic arthritis, Lyme disease, arthritis associated with “vasculitic syndromes”, polyarteritis nodosa, hypersensitivity vasculitis, Luegenec's granulomatosis, polymyalgia rheumatica, joint cell arteritis, calcium crystal deposition arthropathris, pseudo gout, non-articular rheumatism, bursitis, tenosynomitis, epicondylitis (tennis elbow), neuropathic joint disease, hemarthrosis (hemarthrosic), Henoch-Schonlein Purpura, hypertrophic osteoarthropathy, multicentric reticulohistiocytosis, surcoilosis, hemochromatosis, hemoglobinopathy, hyperlipoproteineimia, hypogammaglobulinemia, familial Mediterranean fever, Behat's disease, systemic lupus erythematosus, relapsing fever, multiple sclerosis, septicemia, septic shock, acute respiratory distress syndrome, multiple organ failure, chronic obstructive pulmonary disease, rheumatoid arthritis, acute lung injury, broncho-pulmonary dysplasia. Also, examples of the inflammatory disease include inflammatory skin diseases, such as acute and chronic eczema, atopic dermatitis, contact dermatitis, dermatitis seborrheica, dermatitis exfoliativa, solar dermatitis and psoriasis.

### BRIEF DESCRIPTION OF THE DRAWINGS

FIG. 1 shows a process of isolating a lignan compound from Myristica fragrans.

FIG. 2 shows the ¹³C-NMR spectrum of the inventive lignan compound.

FIG. 3 shows the ¹H-NMR spectrum of the inventive lignan compound.

FIG. 4 shows the ¹H-¹H COSY spectrum of the inventive lignan compound.

FIG. 5 shows the ¹H-¹³C HMBC spectrum of the inventive lignan compound.

FIG. 6 shows the EI-Mass spectrum of the inventive lignan compound.

FIG. 7 shows the cytotoxicity effect of the inventive lignan compound.

FIG. 8 shows analysis results for the NO production-inhibitory effect of the inventive lignan compound.

FIG. 9 shows analysis results for the iNOS expression-inhibitory effect of the inventive lignan compound.

A: Western blot analysis result

B: graph showing iNOS protein levels relative to a control group stimulated by LPS

FIG. 10 shows analysis results for the PGE₂ production-inhibitory effects of the inventive lignan compound (A) and Curcumin(B).

FIG. 11 shows analysis results for the COX-2 expression-inhibitory effect of the inventive lignan compound.

A: Western blot analysis result

B: graph showing COX-2 protein levels relative to a control group stimulated by LPS

FIG. 12 shows analysis results for the TNF-α production-inhibitory effects of the inventive lignan compound (A) and curcumin (B) in macrophages stimulated by LPS.

FIG. 13 shows analysis results for the TNF-α production-inhibitory effects of the inventive lignan compound (A) and indomethacin (B) in human monocyte U937 cells stimulated by P. acnes.

### BEST MODE FOR CARRYING OUT THE INVENTION

Hereinafter, the present invention will be described in detail by examples. It is to be understood, however, that these examples are for illustrative purpose only and are not construed to limit the scope of the present invention. In these examples and test examples, percentages are by weight unless otherwise specified. Activity analysis was repeated at least three times, and the results were expressed as mean ± standard deviation. Also, statistical analysis was performed by Student's t-test, and a value of p<0.05 was considered statistically significant.

### Example 1

### Isolation and Purification of Lignan Compound from Myristica fragrans

<1-1> Isolation and Purification of Lignan Compound

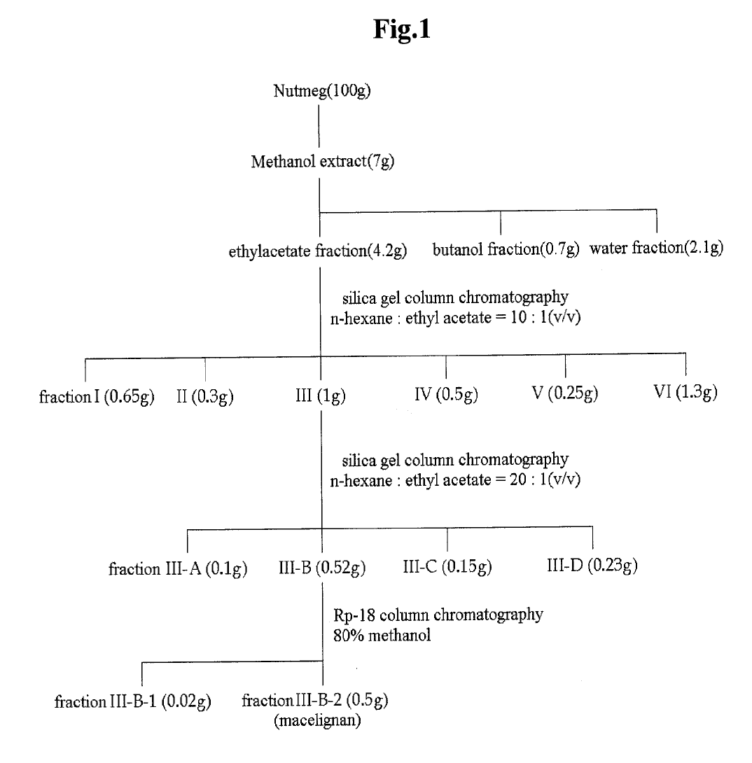

To 100 g (dry weight) of dried and crushed nutmeg, 400 ml of 75-vol % methanol was added, and the solution was left to stand at room temperature for 2 days. The solution was then filtered through Whatman filter paper No. 2. The filtration step was repeated two times. The methanol filtrate was concentrated under vacuum and lyophilized to prepare 7 g of a methanol crude extract of nutmeg. The methanol crude extract was fractionated sequentially with ethyl acetate, butanol and water to obtain 4.2 g of an ethyl acetate fraction. The ethyl acetate fraction was eluted by silica gel column chromatography (Merck Kieselgel 66; 70-230 mesh) with a mixed solvent of hexane and ethyl acetate (10:1 v/v) to obtain 0.1 g of fraction III. The solvent was completely removed with a vacuum rotary evaporator to prepare a crude extract of nutmeg. Then, the fraction III was eluted by silica gel column chromatography (Merck Kieselgel 66; 70-230 mesh) with a mixed solvent of hexane and ethyl acetate (20:1 v/v) to obtain 0.52 g of fraction III-B. The fraction III-B was eluted by Rp-18 column chromatography (Merck LiChroprep; 25-40 μm) with 80% methanol to obtain 0.5 g of single material fraction III-B-2. This isolation process was shown in FIG. 1.

<1-2> Analysis of Structure

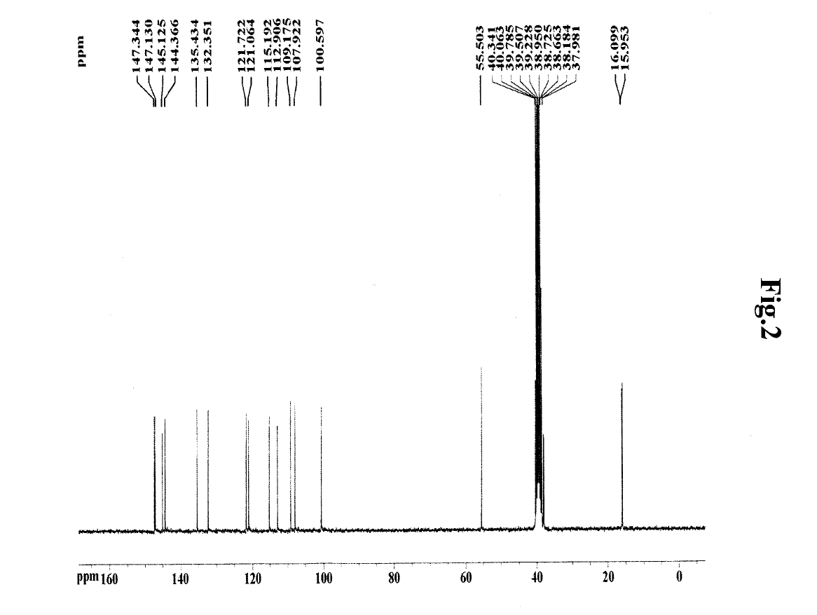

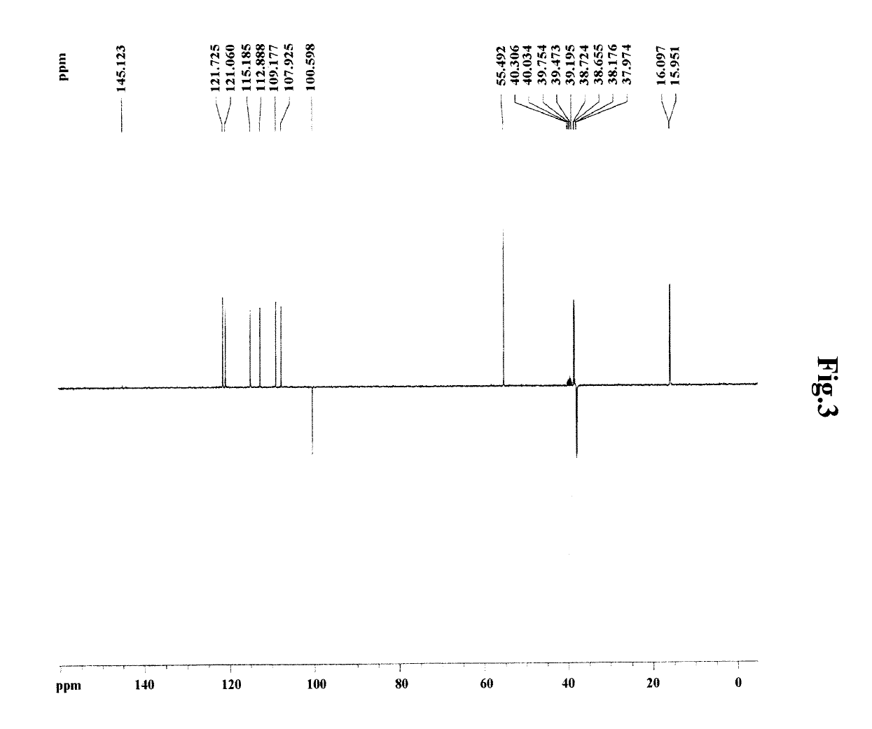

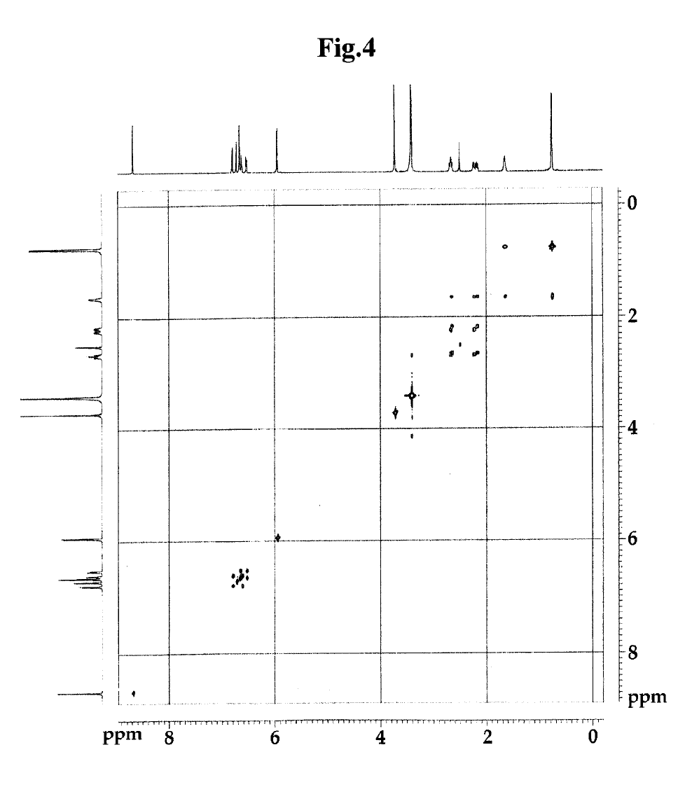

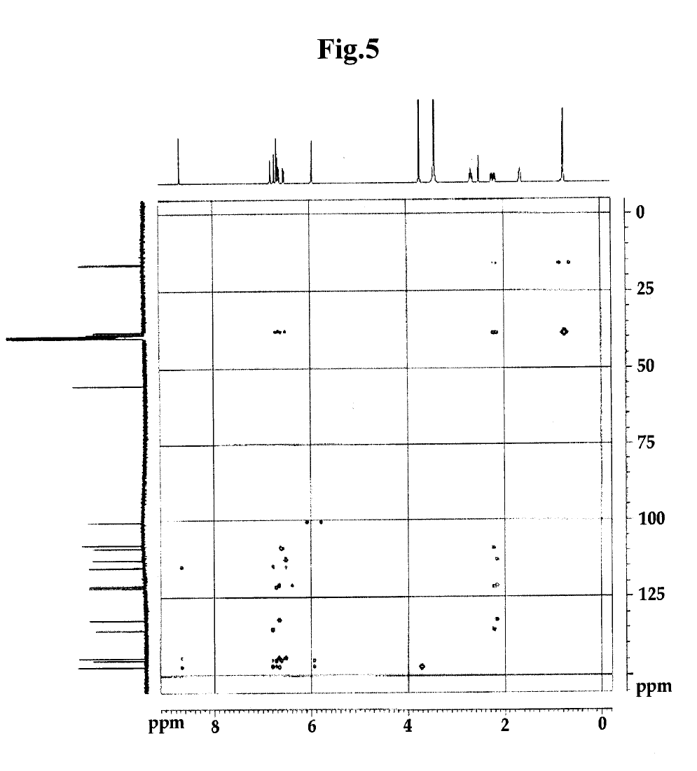

To determine the structure of the isolated single material fraction III-B-2, the ¹H-NMR spectrum and ¹³C-NMR spectrum were analyzed at 600 MHz and 150 MHz, respectively, in DMSO solvent. The results were shown in FIGS. 2 and 3, respectively. To determine ¹H-¹H correlation and ¹H-¹³C correlation on the basis of the results of the ¹³C-NMR and ¹H-NMR spectrum analyses, the ¹H-¹H COSY spectrum and ¹H-¹³C HMBC spectrum were analyzed. The results were shown in FIGS. 4 and 5, respectively. The results of the ¹H-NMR, ¹³C-NMR, ¹H-¹H COSY and ¹H-¹³C HMBC spectrum analyses were collectively analyzed and the results were shown in Table 1 below.

TABLE 1

| Position | ¹³C-NMR | ¹H-NMR | ¹H-¹H COSY | ¹H-¹³C HMBC |
|---|---|---|---|---|
| 1 | 135.4 | | | |
| 2 | 109.2 | 6.72 brs | | C-7, C-6, C-4, C-3 |
| 3 | 147.3 | | | |
| 4 | 145.1 | | | |
| 5 | 107.9 | 6.79 d(7.8) | 6.61 | C-6, C-4, C-3, C-1 |
| 6 | 121.7 | 6.61 dd(7.8) | 6.79 | C-7, C-5, C-4, C-2, C-1 |
| 7 | 38.2 | 2.23 dd(13.2, 9.3) | 1.64, 2.66 | C-8, C-6, C-2, C-1 |
| | | 2.66 dd(13.2, 4.8) | 1.64, 2.23 | C-9, C-8, C-6, C-2, C-1 |
| 8 | 38.7 | 1.64 brs | 0.75, 2.23, 2.66 | C-7 |
| 9 | 16.0 | 0.75 d(6.3) | 1.64 | C-8, C-7 |
| 1′ | 132.4 | | | |
| 2′ | 112.9 | 6.66 brs | | C-7′, C-6′, C-4′, C-3′ |
| 3′ | 147.1 | | | |
| 4′ | 144.4 | | | |
| 5′ | 115.2 | 6.66 d(7.9) | 6.53 | C-6′, C-4′, C-3′, C-1′ |
| 6′ | 121.0 | 6.53 d(7.9, 1.1) | 6.66 | C-7′, C-5′, C-4′, C-2′, C-1′ |
| 7′ | 38.0 | 2.17 dd(13.2, 9.3) | 1.64, 2.66 | C-8′, C-6′, C-2′, C-1′ |
| | | 2.66 dd(13.2, 4.8) | 1.64, 2.17 | C-9′, C-8′, C-6′, C-2′, C-1′ |
| 8′ | 38.7 | 1.64 brs | 0.75, 2.17, 2.66 | C-7′ |
| 9′ | 16.1 | 0.75 d(6.3) | 1.64 | C-8′, C-7′ |
| OMe | 55.5 | 3.72(s) | | |
| O—CH₂—O | 100.6 | 5.95 d(4.8) | | C-3, C-4 |

<1-3> Mass Analysis

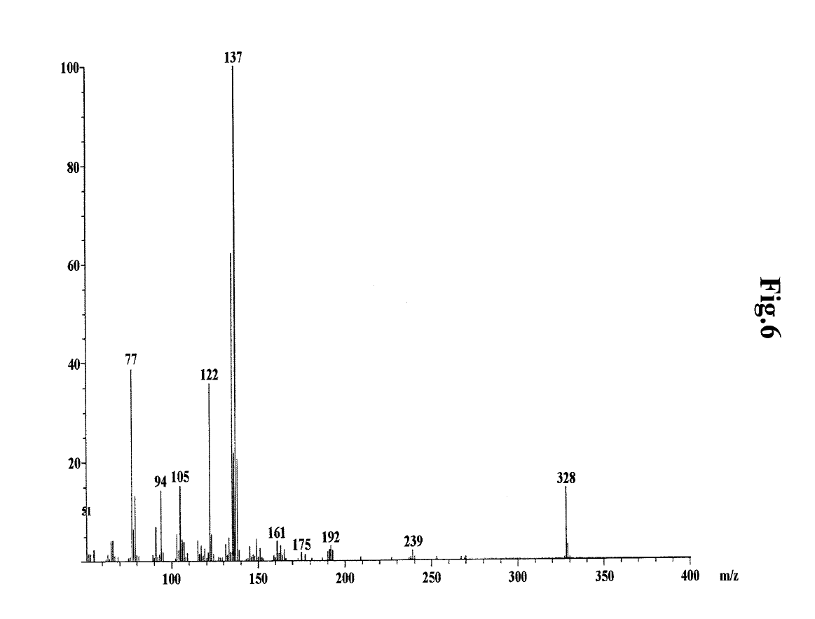

The results of EI/MS conducted to analyze the mass of the above-isolated single material were shown in FIG. 6. In the EI/MS analysis, [M]⁺was observed at m/z 328, indicating that the isolated compound has a molecular weight of 328 and a molecular formula of C₂₀H₂₄0₄.

The results of the ¹H-NMR, ¹³C-NMR, ¹H-¹H COSY, ¹H-¹³C HMBC and EI/MS spectrum analyses were analyzed comparatively with the previously reported study results (Woo, W. S. et al., Phytochemistry, 26: 1542-1543, 1987). As a result, it was found that the isolated single material was macelignan represented by Chemical Formula 1:

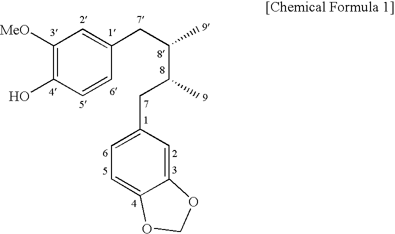

### Example 2

### Examination of Cytotoxicity Effect of Inventive Lignan Compound

<2-1> Culture of RAW264.7 Cell Line

In order to examine the effect of macelignan obtained in <Example 1> on the production of inflammatory response mediators, the macrophage RAW264.7 cell line was used. The macrophage RAW264.7 cell line(ATCC TIB-71) was purchased from American Tissue Culture Collection (Rockville, Md., USA). The cell line was cultured in DMEM (Dulbecco's Modified Eagle's Medium, Gibco, USA) supplemented with 10% heat inactivated FBS (fetal bovine serum, Gibco, USA), 100 U/ml penicillin G and 100 μg/ml streptomycin, in a 5% CO₂ incubator at 37° C.

<2-2> Measurement of Cytotoxicity

In order to examine the effect of the inventive macelignan on the viability of RAW 264.7 cells, analysis was performed based on the reduction of MTT changed into a purple formazan product by mitochondrial dehydrogenase (Hayon T. et al., Leuk. Lymphoma. 44(11): 1957-1962, 2003). 1×10⁶ cells/ml of RAW264.7 cells were inoculated into RPMI 1640 medium, and after 6 hours, were treated with the inventive macelignan at concentrations up to 1-80 μM/ml. After 24 hours, the viability of the cells was measured by the MTT assay.

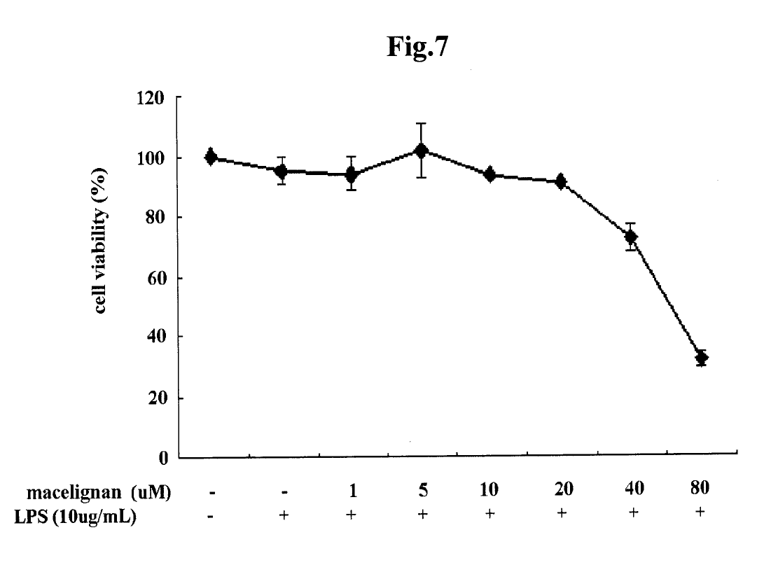

As a result, as shown in FIG. 7, the inventive macelignan had no significant effect on the viability of the RAW264.7 cells at macelignan concentrations of 1-20 μM. Based on these results, 1-20 μM concentrations of macelignan were used in a subsequent inflammation test.

### Example 3

### Examination of NO-Inhibitory Effect of Inventive Lignan Compound

<3-1> Inhibition of NO Production

Macrophages stimulated by IFN-γ or LPS highly express iNOS to produce a large amount of inflammatory response mediator NO (Miyasaka and Hirata., Immunol. Today., 16: 128-130, 1995; Guzik et al., J. Physiol. Pharmacol., 54(4): 469-487, 2003). Accordingly, whether the inventive macelignan has any effect on NO production in RAW264.7 cells activated with LPS was examined.

RAW264.7 cells were diluted at a concentration of 1×10⁶ cell/ml and then inoculated into RPMI 1640 medium. After 5 hours, the inventive macelignan was added to the medium at each of a concentration of 1-20 μM, followed by incubation for 2 hours. Then, the medium was treated with LPS (10 μg/ml) and incubated for 24 hours. A control group was treated only with LPS. The production of NO was quantified by measuring NO₂⁻, a reaction product of NO, using the remains of cell culture(Han et al., Life Sci., 75(6): 675-684, 2004). 100 μl of the remains of cell culture and the same volume of Greiss reagent (0.5% sulfanilyamide, 0.05% N-(1-naphthyl) ethylene diamine dihydrochloride/2.5% H₃PO₄) were mixed with each other on a 96-well tissue culture plate and allowed to react in a dark place for 10 minutes. Then, the absorbance of the sample was measured at 550 nm using the ELISA microplate reader. The concentration of N0₂⁻ was plotted as a standard curve using NaNO₃, and the production of NO was determined comparable to the standard curve. All tests were repeated at three times, and then quantified by student's t-test.

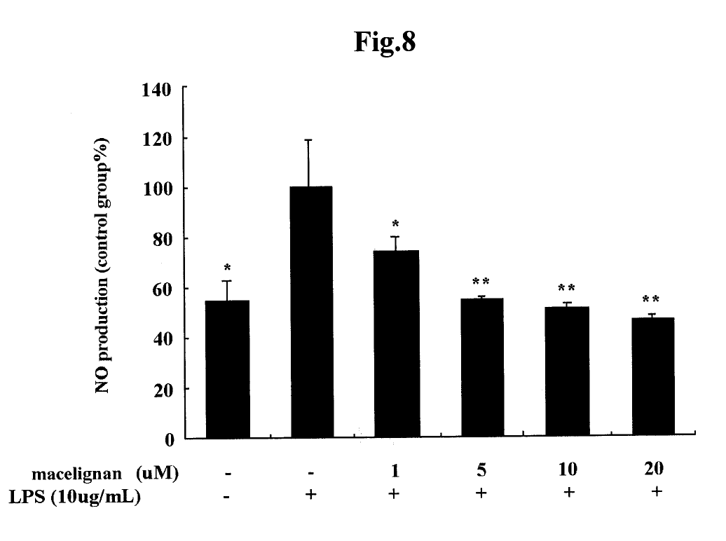

As a result, as shown in FIG. 8, the production of NO was greatly increased as a result of treatment with LPS alone, but it was concentration-dependently inhibited by treatment with the inventive macelignan. Particularly, it could be observed that the inventive macelignan had excellent effects on the inhibition of NO production even at low concentrations of 1 μM and 5 μM (P<0.01). Also, in the case of treatment with 20 μM of macelignan, the production of NO was inhibited to an extent almost similar to that of the group treated with nothing.

<3-2> Inhibition of iNOS Expression

If macrophages are stimulated by LPS, iNOS will be highly expressed while producing a large amount of NO. Accordingly, in order to examine the relationship between the NO producing inhibition of the inventive macelignan confirmed in Example <3-1> and the iNOS, the effect of the macelignan on the expression of iNOS was measured.

For this purpose, RAW 264.7 cells treated with the inventive macelignan and LPS were dissolved and the protein was quantified by the Bradford assay. 10 μg of the protein was separated on 10% SDS-PAGE, and then transferred to a nitrocellulose membrane by a transfer solution (20% methanol, 25 mM Tris, 192 mM glycine, pH 8.3) (Hall, Methods Mol. Biol., 261: 167-174, 2004). The nitrocellulose membrane was brought into close contact with SDS-polyacrylamide gel, and then placed in a mini-gel transfer kit. Then, the sample was loaded into the kit and electrophoresed at 100 V for 1 hour. Next, the membrane was washed one time with TBST (Tris buffered saline Tween-20) solution and dried on dry filter paper at room temperature. To eliminate non-specific reactions, the membrane was left to stand while sufficiently shaking it with 5% containing non-fat skim milk in TBST solution at 4° C. for at least 24 hours. Then, the membrane was washed three times with TBST solution and injected with an anti-iNOS antibody (1:2,000) (Calbiochem) and allowed to react at room temperature for 1 hour. Then, the membrane was washed three times with TBST solution for 10 minutes each washing time. The washed membrane was injected with anti-rabbit IgG-HRP conjugated with HRP(horse radish peroxidase) (1:2,000) (Calbiochem) and allowed to react on a shaker for 1 hour. Then, the membrane was washed three times with TBST solution, after which it was immersed in ECL (enhanced chemiluminescence) solution and evenly wetted with the solution while shaking it for 1 minute. The ECL solution was prepared by mixing solution A (containing luminol and enhancer) with solution B (containing hydrogen peroxide) in the same amount and shaking the mixed solution well for 1 minute. The membrane was taken out from the ECL solution, dehydrated and then scanned with X-ray films in a dark room.

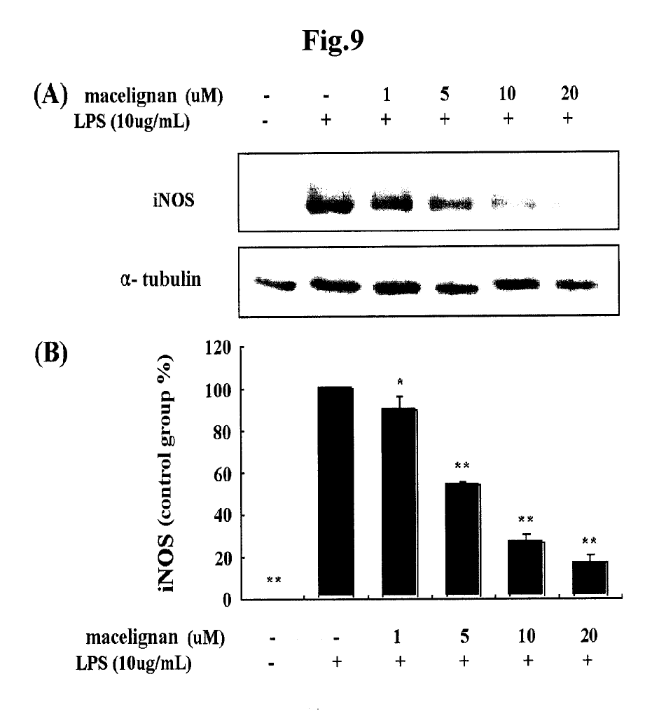

As a result, as shown in FIG. 9, the inventive macelignan concentration-dependently inhibited the expression of iNOS in macrophages and showed a remarkable inhibitory effect starting with a concentration of 5 μM (P<0.01).

From the above results, it could be found that the inventive macelignan not only inhibits the production of inflammation-inducing factor NO, but also inhibits the expression of iNOS that produces NO.

### Example 4

### Examination of COX-2-Inhibitory Effect of Inventive Lignan Compound

<4-1> Inhibition of PEG2 Production

Similarly to the fact that iNOS has a close connection with inflammation reactions, it is known that COX-2 is an enzyme necessary for the production of prostaglandins (PG) mediating inflammatory reactions, and the expressions and activities of iNOS and COX have a connection with each other (Surh et al., Mutat. Res., 481: 243-268, 2001). Accordingly, whether the inventive macelignan has any effect on the production of PGE₂ in macrophages activated by LPS was examined.

First, 1×10⁶ cells/ml of RAW264.7 cells were inoculated into a 96-well tissue culture plate and left to stand at room temperature for 5 hours. Then, the inventive macelignan was added to the cells at each of concentrations of 1-20 μM and incubated for 2 hours. A negative control group was not treated with anything, and a positive control group was treated with curcumin (isolated from Curcuma longa; Sigma) reported to have PGE₂ inhibitory activity. Then, the cells were treated with 1 μg/ml of LPS and cultured for 18 hours. The production of PGE₂ in the macrophages was quantified by an assay kit (R&D System Inc, Minneapolis, USA) using an ELISA method (Chen et al., Biochem. Pharmacol., 68: 1089-1100, 2002).

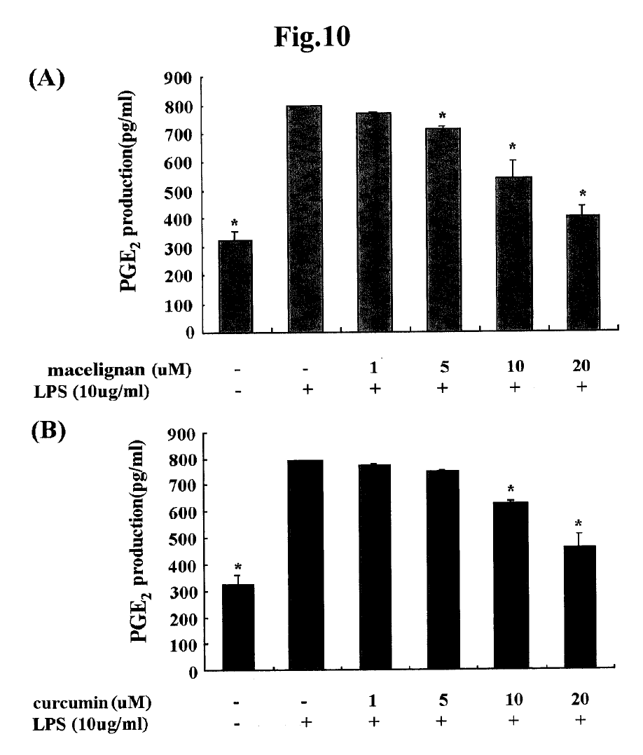

As a result, as shown in FIG. 10, it was observed that the production of PGE₂ was greatly increased as a result of treatment with LPS alone, but was concentration-dependently inhibited by treatment with the inventive macelignan. This inhibitory effect was shown even at a macelignan concentration of 5 μM. This PGE₂ production-inhibitory effect of the inventive macelignan was almost similar to the case of treatment with curcumin, and showed the same pattern as the NO and iNOS inhibitory effect confirmed in <Example 3> (P<0.05).

<4-2> Inhibition of COX-2 Expression

The present inventors examined the expression of COX-2 having a direct effect on the production of PGE₂ by Western blot analysis. It was performed in the same manner as described in Example <3-2>, except that an anti-COX-2 antibody (1:2,000) (Calbiochem) was used as a primary antibody, and anti-goat IgG-HRP (1:2,000) (Calbiochem) was used as a secondary antibody.

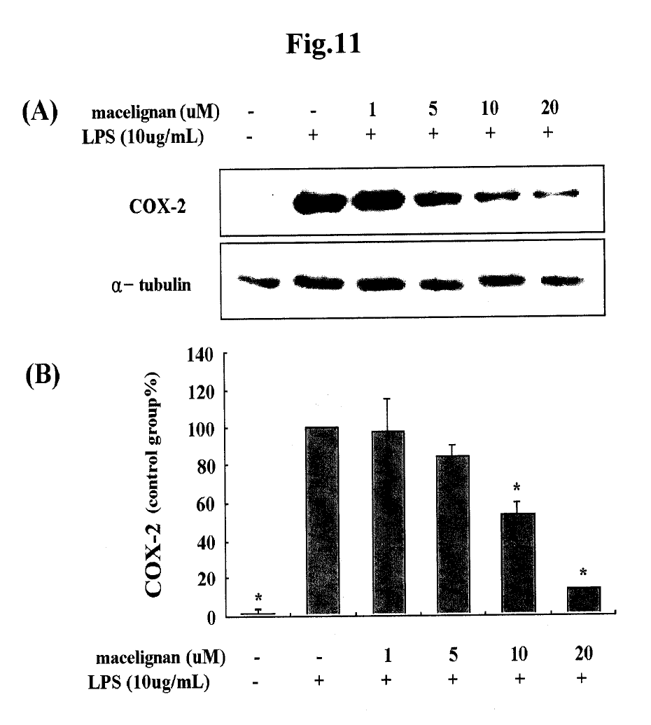

As a result, as shown in FIG. 11, the inventive macelignan inhibited the expression of the COX-2 protein in a concentration-dependent manner. Particularly at macelignan concentrations of 10-20 μM, the expression level of the COX-2 protein was significantly reduced.

From the above results, it could be found that the inventive macelignan not only inhibits the production of inflammation-inducing factor PEG₂, but also inhibits the expression of COX-2 that produces PEG₂.

### Example 5

### Examination of TNF-α Inhibitory Effect of Inventive Lignan Compound

TNF-α is an inflammatory cytokine which plays an important role in inflammatory reactions. Accordingly, the effect of the inventive macelignan on the production of TNF-α was examined.

<5-1> Inhibition of TNF-α Production in Macrophages

First, 1×10⁶ cell/ml of RAW264.7 cells were inoculated into a 96-well tissue culture plate and left to stand at room temperature for 5 hours. Then, the inventive macelignan was added to the cells at each of a concentration of 1-20 μM and incubated for 2 hours. A negative control group was not treated with anything, and a positive control group was treated with curcumin (Sigma) (Araujo and Leon, Mem. Inst. Oswaldo. Cruz., 96(5): 723-728, 2001; Chainani-Wu, J. Altern. Complement., 9(1): 161-168, 2003). Then, the cells were treated with 1 μg/ml of LPS and cultured for 18 hours. The production of TNF-α in the macrophages was quantified with an assay kit (R&D System Inc, Minneapolis, USA) using an ELISA method (Chen et al., J. Dermatol. Sci. 29: 97-103, 2002).

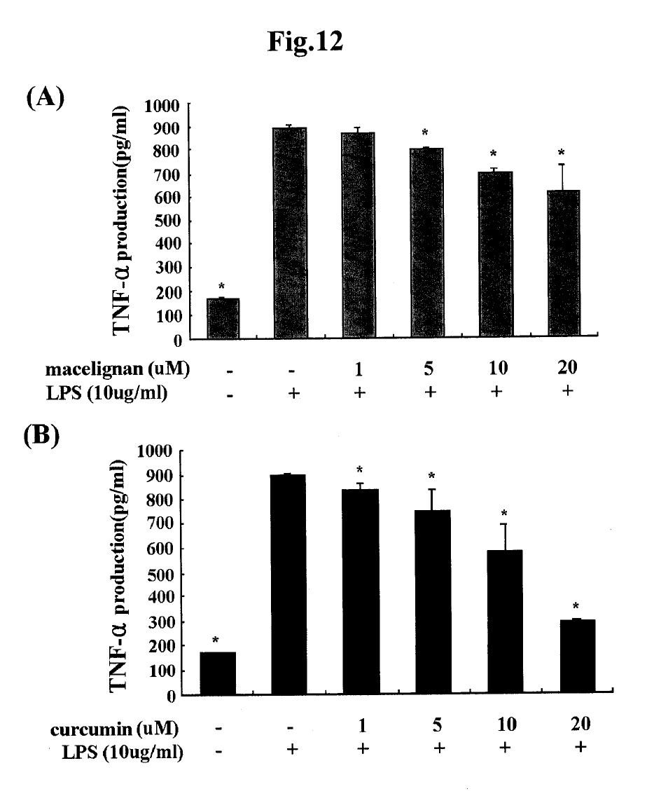

As a result, as shown in FIG. 12, the production of TNF-α was significantly reduced starting with a macelignan treatment concentration of 5 μM (P<0.05).

<5-2> Inhibition of TNF-α Production in Human Monocytic Cells

By the present inventors, the production of TNF-α in human monocytic U937 cells activated with Propionibacterium acnes was measured in the same manner as in Example <5-1>. However, a positive control group was treated with indomethacin (Sigma) (Walch and Morris, Endocrinology. 143(9): 3276-3283, 2002).

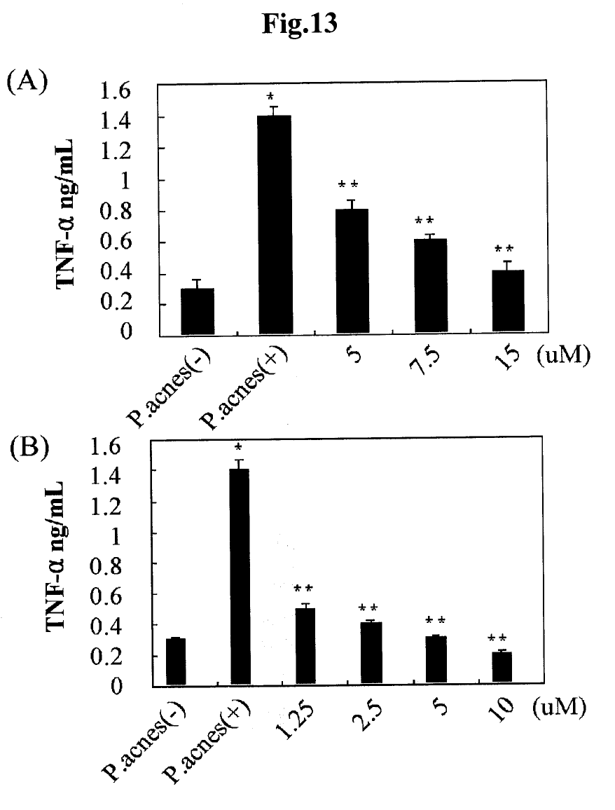

As a result, as shown in FIG. 13, it could be observed that the production of TNF-α in the human monocytic cells was reduced by the inventive macelignan in a concentration-dependent manner (P<0.01).

From the above results, it could be seen that the inventive macelignan inhibited the production of TNF-α that induced and/or mediated acute inflammation and systemic inflammatory reactions.

### Example 6

### Examination of Anti-Inflammatory Activity of Inventive Lignan Compound in Animal Model

The anti-inflammatory activity of the lignan compound isolated and purified in <Example 1> was tested in animal models. The anti-inflammatory activity was measured by edema inhibition test on rats. As the test animals, 5-week-old Wistar rats (DaeHan Biolink Co., Ltd, Korea) were used. The animals were provided with standard pellet forming rat feed (Cheiljedang Corporation, Korea) and given freely to feed and water. Also, the animals were housed in conditions of 12-hr light/12-hr dark cycle, temperature of 25±2° C. and humidity of 60±10%. Inflammation inducing agent TPA (12-O-tetradecanoylphorbol-13-acetate; Sigma) was dissolved in acetone to a concentration of 200 μg/mL. The edemas of the rat ears were induced by applying the TPA solution locally to each of the outer and inner faces of the ear in an amount of 10 μl/ear (4 μg/ear). The macelignan purified in <Example 1> and non-steroidal anti-inflammatory drug indomethacin as a control substance were dissolved in acetone and used in amounts of 20, 200 and 2000 μg/ear. Each of the macelignan and the indomethacin was applied locally to the rat ears at 30 minutes after treatment with TPA. A control group was locally applied with acetone. The thickness of the rat ears was measured with a caliper 8 hours after treatment with each of the substances. An increase in the ear thickness in the group treated with the sample was compared to that of the group untreated with the sample, and inflammation inhibitory effect was measured by calculating percent edema inhibition. The results were shown in Table 2 below.

TABLE 2

| Drug | Number | Dose | Edema thickness | Edema inhibition |
|---|---|---|---|---|
| administered | of rats | (μg/ear) | (μm) | (%) |
| Control | 20 | 0 | 248 ± 8 | |
| Inventive | 20 | 20 | 157 ± 9* | 36.7 |
| macelignan | 20 | 200 | 98 ± 6* | 60.5 |
| | 20 | 2000 | 52 ± 4* | 79.0 |
| indomethacin | 20 | 20 | 185 ± 5* | 25.0 |
| | 20 | 200 | 108 ± 8* | 56.5 |
| | 20 | 2000 | 64 ± 7* | 74.2 |

*p < 0.01

### Example 7

### Preparation of Macelignan-Comprising Creams and Examination of Anti-Inflammatory Activities Thereof

<7-1> Preparation of Creams Comprising Macelignan

Using the inventive macelignan, each of creams having various compositions shown in Table 3 below was prepared. First, substances indicated as “B” in Table 3 were dissolved at 75-80° C. Also, among substances indicated as “C” in Table 3, cetyl alcohol and a preservative were dissolved at the same temperature as above. The substances indicated as “C” were emulsified in the substances indicated as “B”. Then, the inventive macelignan indicated as “A” in Table 3 was added to the emulsions at each of concentrations of 5.0, 0.5, 0.05 and 0.005%. Finally, a fragrance was added and the balance of purified water was then added, thus preparing creams.

TABLE 3

| 1 | | | 2 | 3 | 4 |
|---|---|---|---|---|---|
| Main | A | 5.0% macelignan | 0.5% macelignan | 0.05% macelignan | 0.005% |
| components | | | | | macelignan |
| | B | 2.0% glycerin | 2.0% glycerin | 2.0% glycerin | 2.0% glycerin |
| | | 2.0% propylene | 2.0% propylene | 2.0% propylene | 2.0% propylene |
| | | glycol | glycol | glycol | glycol |
| | | 8.0% chloride | 8.0% chloride | 8.0% chloride | 8.0% chloride |
| | | lauryl sulfide | lauryl sulfide | lauryl sulfide | lauryl sulfide |
| | | 5.4% stearin | 5.4% stearin | 5.4% stearin | 5.4% stearin |
| | | 4.5% mineral oil | 4.5% mineral oil | 4.5% mineral oil | 4.5% mineral oil |
| | C | 0.02% fragrance | 0.02% fragrance | 0.02% fragrance | 0.02% fragrance |
| | | 6.5% cetyl alcohol | 6.5% cetyl alcohol | 6.5% cetyl alcohol | 6.5% cetyl alcohol |
| | | Balance purified | Balance purified | Balance purified | Balance purified |
| | | water | water | water | water |
| | | 0.1% preservative | 0.1% preservative | 0.1% preservative | 0.1% preservative |

<7-2> Examination of Anti-Inflammatory Activity

The anti-inflammatory activities of the macelignan-comprising creams prepared in Example <7-1> were measured through edema inhibition test on rats. The edema inhibition test was performed in the same manner as in <Example 6>. The results were shown in Table 4 below.

TABLE 4

| | Sample | | | |
|---|---|---|---|---|
| | 1 | 2 | 3 | 4 |
| Macelignan content | 0.005% | 0.05% | 0.5% | 5% |
| Edema inhibition (%) | 14.2 | 42.3 | 64.6 | 88.7 |

From the results in Table 4, it could be seen that the creams comprising the inventive macelignan inhibited the rat edema induced by TPA in a manner dependent on the concentration of the macelignan comprised in each of the cream.

### Example 8

### Examination of Anti-Inflammatory Activity of Myristica fragrans Extract

<8-1> Preparation of Methanol Extract

To 100 g (dry weight) of dried and crushed nutmeg, 400 ml of 95-vol % methanol was added and left to stand at room temperature for 2 days. The solution was filtered through Whatman filter paper No. 2. The filtration step was repeated two times. The methanol filtrate was concentrated under vacuum and lyophilized to obtain 16.2 g of a methanol crude extract.

<8-2> Preparation of Hexane Extract

To 100 g (dry weight) of dried and crushed nutmeg, 400 ml of 100-vol % hexane was added and left to stand at room temperature for 2 days. The solution was filtered through Whatman filter paper No. 2. The filtration step was repeated two times. The hexane filtrate was concentrated under vacuum and lyophilized to obtain 37.0 g of a hexane crude extract.

<8-3> Examination of Anti-Inflammatory Activity of Myristica fragrans Extract in Animal Model

The anti-inflammatory activities of the Myristica fragrans extracts prepared in Example <8-1> and Example <8-2> were tested in animal models. The anti-inflammatory activities were measured by performing edema inhibition test on rats in the same manner as in <Example 6>. The results were shown in Table 5 below.

TABLE 5

| Drug | Number | Dose | Edema thickness | Edema inhibition |
|---|---|---|---|---|
| administered | of rats | (μg/ear) | (μm) | (%) |
| Control | 20 | 0 | 248 ± 8 | |
| Methanol crude | 20 | 50 | 198 ± 3* | 20.2 |
| extract | 20 | 500 | 119 ± 8* | 52.0 |
| | 20 | 000 | 83 ± 10* | 66.5 |
| Hexane crude | 20 | 50 | 207 ± 4* | 16.5 |
| extract | 20 | 500 | 146 ± 9* | 41.1 |
| | 20 | 5000 | 106 ± 7* | 57.3 |

*p < 0.01

As shown in Table 5, it could be observed that the inventive methanol crude extract and hexane crude extract of Myristica fragrans all inhibited the rat edema induced by TPA in a concentration-dependent manner (statistical significance p<0.01).

### Preparation Example 1

### Preparation of Pharmaceutical Formulations Comprising Inventive Pharmaceutical Composition for Treating or Preventing Inflammatory Disease

<1-1> Preparation of Tablet Formulation

25 mg of the inventive lignan compound or Myristica fragrans extract, 26 mg of lactose for direct tableting, 3.5 mg of Avicel (microcrystalline cellulose), 15 mg of disintegration aid sodium starch glyconate and 8 mg of binder L-HPC (low-hydroxypropylcellulose) for direct tableting were placed and mixed with each other in U-type mixer for 20 minutes. After completion of the mixing, 1 mg of lubricant magnesium stearate was further added thereto and mixed for 3 minutes. The mixture was subjected to test for quantitative analysis and moisture content analysis, tableted and coated with a film, thus preparing a tablet formulation.

<1-2> Preparation of Syrup

A syrup comprising 2% (w/v) of the inventive macelignan or its pharmaceutically acceptable salt as an active ingredient was prepared in the following manner:

2 g of an acid addition salt of the inventive macelignan, 0.8 g of saccharin and 25.4 g of sugar were dissolved in 80 g of hot water. The solution was cooled, to which 8.0 g of glycerin, 0.04 g of fragrance, 4.0 g of ethanol, 0.4 g of sorbic acid and a suitable amount of distilled water were then added. To the mixture, water was added to make a volume of 100 ml.

<1-3> Preparation of Capsule Formulation

50 mg of the inventive lignan compound or Myristica fragrans extract, 50 mg of lactose, 46.5 mg of starch, 1 mg of talc and a suitable amount of magnesium stearate were mixed with each other. The mixture was filled in a hard gelatin capsule, thus preparing a capsule formulation.

<1-4> Preparation of Injectable Liquid

An injectable liquid comprising 10 mg of the active ingredient was prepared in the following manner:

1 g of a hydrochloride of the inventive macelignan, 0.6 g of sodium chloride and 0.1 g of ascorbic acid were dissolved in distilled water to make 100 ml of a solution. The solution was bottled and sterilized by heating it at 20° C. for 30 minutes.

### Application Example 1

### Gastric Inflammatory Digestive Diseases

It was known that gastric inflammation is mainly caused by Helicobacter pylori infection, although various external factors and irregular eating habits are involved therein. Helicobacter pylori causes not only gastric ulcer and gastritis, but also gastric cancer. During the proliferation of Helicobacter pylori, enzyme COX-2 (cyclooxygenase-2) also increases at the same time (Nam N. T. et al., Clin. Cancer Res. 10(23): 8105-8113, 2004). It was known that, when infected with Helicobacter pylori, gastric mucosal cells proliferate into cancer cells; COX-2 inhibitors suppress the growth and proliferation of gastric mucosal cells into cancer cells and inhibit normal tissue from changing into cancer tissue. It was found that a group administered with the COX-2 inhibitor is superior to a group administered with no COX-2 inhibitor in the effect of killing cancer tissue by an apoptosis method (Nam N. T. et al., Clin. Cancer Res. 10(23): 8105-8113, 2004). Accordingly, the COX-2 inhibitory effect of the inventive lignan compound suggests that the inventive lignan compound has a sufficient therapeutic effect, because it helps to treat gastric inflammation so as to be able to prevent gastric cancer in an early stage.

### Application Example 2

### Arthritis

Arthritis is caused by autoimmune abnormality, but chronic inflammation occurring in the synovial cavity between joints during the progression of arthritis induces angiogenesis so as to destroy cartilage. Arthritis includes infectious arthritis, degenerative arthritis, rheumatoid arthritis, and arthritis caused by avascular necrosis of femoral head, ankylosing spondylitis and congenital malformation. Regardless of the cause of arthritis, the chronic inflammation formed in the synovial cavity between joints during the progression of arthritis is known to induce angiogenesis and is characterized by invading joints with a new capillary vessel to cause damage to cartilage (Kocb A. E. et al., Arth. Rheum., 29:471-479, 1986; Stupack D. G. et al., J. Med. Biol. Rcs., 32:578-281, 1999; Koch A. E., Arthritis Rheum., 41:951-962, 1998). In this case, it is reported that an inflammatory response, which occurs in several steps depending on the kind of diseases to destroy cartilage, plays an important role in the progression of the disease, and the formation of angiogenesis into joints acts as an important pathological mechanism (Colville-Nash, P. R. et al., Ann. Rheum. Dis., 51, 919-925, 1992; Eisenstein, R., Pharmacol. Ther., 49:1-19, 1991). For the treatment of arthritis, it is preferred to inhibit pain and a state of inflammation rather than to treat by causing, so as to reduce the destruction rate of joints or muscles and minimize the loss of their function. Accordingly, the inventive lignan compound or Myristica fragrans extract is highly effective in the prevention of arthritis progression and in the treatment of arthritis.

### INDUSTRIAL APPLICABILITY

As described above, the inventive lignan compound has the effect of inhibiting inflammation reactions by inhibiting the production or expression of inflammation mediators NO, iNOS, PGE₂, COX-2 and TNF-α. Accordingly, the inventive lignan compound or Myristica fragrans extract will be highly useful for the treatment or prevention of inflammatory diseases.

1. A pharmaceutical composition for the treatment or prevention of an inflammatory disease, which comprises a lignan compound represented by Formula I or a pharmaceutically acceptable salt thereof: 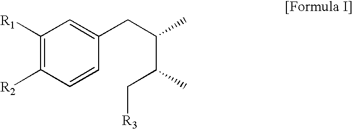
wherein R₁ and R₂ are each independently a C₁₋₅ alkoxy group or a hydroxyl group, and R₃ is

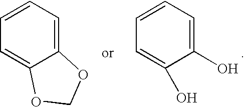

2. The pharmaceutical composition of claim 1, wherein R₁ is a methoxy group, R₂ is a hydroxyl group, and R₃ is

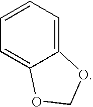

3. A pharmaceutical composition for the treatment or prevention of an inflammatory disease, which comprises water or a C₁-C₆ organic solvent extract of Myristica fragrans as an active ingredient.

4. The pharmaceutical composition of claim 1 or 3, wherein the inflammatory disease is any one selected from the group consisting of inflammatory bowel disease, peritonitis, osteomyelitis, cellulitis, pancreatitis, trauma causing shock, bronchial asthma, allergic rhinitis, cystic fibrosis, acute bronchitis, chronic bronchitis, acute bronchiolitis, chronic bronchiolitis, osteoarthritis, gout, spondyloarthropathy, ankylosing spondylitis, Reiter's syndrom, psoriatic arthropathy, spondylitis associated with inflammatory bowel disease , juvenile arthropathy, juvenile ankylosing spondylitis, reactive arthropathy, infectious arthritis, post-infectious arthritis, gonococcal arthritis, tuberculous arthritis, viral arthritis, fungal arthritis, syphilitic arthritis, Lyme disease, arthritis associated with “vasculitic syndromes”, polyarteritis nodosa, hypersensitivity vasculitis, Luegenec's granulomatosis, polymyalgia rheumatica, joint cell arteritis, calcium crystal deposition arthropathris, pseudo gout, non-articular rheumatism, bursitis, tenosynomitis, epicondylitis (tennis elbow), neuropathic joint disease, hemarthrosis (hemarthrosic), Henoch-Schonlein Purpura, hypertrophic osteoarthropathy, multicentric reticulohistiocytosis, surcoilosis, hemochromatosis, hemoglobinopathy, hyperlipoproteineimia, hypogammaglobulinemia, familial Mediterranean fever, Behat's disease, systemic lupus erythematosus, relapsing fever, multiple sclerosis, septicemia, septic shock, acute respiratory distress syndrome, multiple organ failure, chronic obstructive pulmonary disease, rheumatoid arthritis, acute lung injury, broncho-pulmonary dysplasia and inflammatory skin disease.

5. A method for preventing or treating an inflammatory disease, comprising administering to a subject in need thereof an effective amount of a lignan compound represented by Formula I or a pharmaceutically acceptable salt thereof: 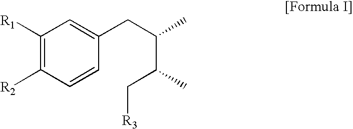
wherein R₁ and R₂ are each independently a C₁₋₅ alkoxy group or a hydroxyl group, R₃ is

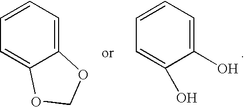

6. The method of claim 5, wherein R₁ is a methoxy group, R₂ is a hydroxyl group, and R₃ is

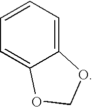

7. A method for preventing or treating an inflammatory disease, comprising administering to a subject in need thereof an effective amount of water or a C₁-C₆ organic solvent extract of Myristica fragrans.

8. Use of a lignan compound represented by Formula I for preparing a pharmaceutical composition for the prevention or treatment of an inflammatory disease: 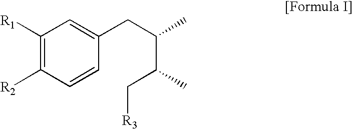
wherein R₁ and R₂ are each independently a C₁₋₅ alkoxy group or a hydroxyl group, R₃ is

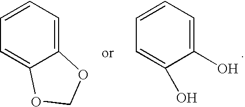

9. The use of claim 8, wherein R₁ is a methoxy group, R₂ is a hydroxyl group, and R₃ is

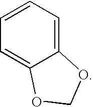

10. Use of water or a C₁-C₆ organic solvent extract of Myristica fragrans for preparing a pharmaceutical composition for the prevention or treatment of an inflammatory disease.
